(19) United States
(12) Patent Application Publication
(10) Pub. No.: US 2003/0229380 A1
(43) Pub. Date: Dec. 11, 2003

(54) Heart failure therapy device and method

(76) Inventors: John M. Adams, Sammamish, WA (US); Clifton A. Alferness, Redmond, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/287,254

(22) Filed: Oct. 31, 2002

(51) Int. Cl. A61N 1/18

(52) U.S. Cl. 607/9

(57) ABSTRACT

The heart rate of a patient with conditions such as chronic heart failure, ischemia, or acute myocardial infarction is reduced by electrically stimulating the right vagus nerve of the patient. A lead is implanted with electrodes in electrical communication with tissue proximate to the vagus nerve. A stimulator in electrical communication with the electrodes delivers electrical energy that stimulates the release of acetylcholine from the vagus nerve. The amount of energy may be determined in accordance with a difference between the patient's actual heart rate and a maximum target heart rate for the patient. Delivery of energy to the lead electrodes is preferably synchronized with the detection of a P-wave. Automatic adjustment of the target heart rate may be based on current day and/or time of day information, and patient physical activity. The voltage, pulse width, or number of pulses in the stimulation may be controlled.

### FIELD OF THE INVENTION

The present invention relates to methods and apparatus that provide therapy to patients suffering cardiovascular disease, and more particularly, to therapy for patients experiencing elevated heart rates due to chronic heart failure or ischemia.

### BACKGROUND OF THE INVENTION

Heart failure is a syndrome resulting from cardiac dysfunction. Typically, a patient suffering from heart failure experiences breathlessness, fatigue, and fluid retention. The heart is unable to efficiently deliver an adequate amount of blood, and hence oxygen and other nutrients, throughout the body. The patient's cardiac dysfunction may be acute or chronic.

Heart failure can be caused by a variety of diseases and conditions. For example, heart failure may result from ischemic heart disease in which blockage of coronary arteries causes injury and irreversible damage to the heart muscle. Heart failure may also result from hypertension, diseases of the heart valves, cardiomyopathy, and congenital heart diseases, among other possible causes. Chronic heart failure, without treatment, has a high mortality rate.

Prior art approaches to treating heart failure typically include the administration of drugs, such as acetylsalicylic acid (aspirin), beta-blockers, and ACE inhibitors. Aspirin is known to provide a mild blood diluting effect. Beta-blockers are known to result in an improved cardiac output while at the same time reducing the heart rate. ACE inhibitors are known to prevent the formation of a hormone that makes blood vessels contract, resulting in a lower pressure that is needed for the heart to pump blood.

As a patient's heart weakens or fails, its ability to pump blood diminishes, and cardiac output falls. A failing heart or weak heart typically compensates for the reduced output by beating at a faster rate. However, during each cardiac contraction, the cardiac muscle itself is not perfused with oxygenated blood to replenish its own energy needs. Cardiac muscle is perfused with blood during the time between heart contractions. An increased heart rate places more demand on the heart muscle while at the same time provides less time for perfusion of blood through the coronary arteries. Thus there is a need to slow the heart rate of patients in these patient groups.

Electrical stimulation of a patient's vagus nerves is known to influence the activity of the sinoatrial (SA) node and the atrioventricular (AV) node of the patient's heart, and hence influence the patient's heart rate. Specifically, stimulation of the right vagus nerve is known to slow the rate of excitation of the SA node (which operates as the heart's natural pacemaker). Generally, the literature reports the effect of stimulating the right vagus nerve as a sudden and dramatic slowing of the heart rate. While no widespread commercial use has been made of stimulating the vagus nerves, the literature describes several attempts that have been made. The focus of these efforts has uniformly been either to stop supra-ventricular arrhythmias (such as atrial fibrillation or atrial tachycardia) or to ameliorate the effect of these arrhythmias on the ventricular rate. In particular, these techniques attempted to control the ventricular rate in the presence of a supra-ventricular arrhythmia by stimulating the vagus nerves (typically the left vagus nerve) to increase the magnitude of AV delay. For instance, U.S. Pat. No. 5,916,239 discloses stimulation of the left vagus nerve to slow the ventricular response to atrial fibrillation. The device disclosed in the '239 patent, however, does not address patients having a normal sinus rhythm with an elevated heart rate due to heart failure or ischemia.

What is needed is a device and method that reduces the heart rate of a patient suffering from cardiovascular disease so that the heart will have a longer time to receive oxygenated blood and nutrients between heart beats, and hence maintain its strength. A gradual slowing of the heart rate may be needed because a rapid decrease in heart rate may not allow the heart to compensate adequately with increased stroke volume and may cause the person to faint due to a sudden, inadequate supply of blood to the organs. The present invention addresses these needs and other shortcomings in the prior art.

### SUMMARY OF THE INVENTION

The present invention provides a method and apparatus for reducing the heart rate of a patient having a normal sinus heart rhythm. The present invention is particularly beneficial to patients with an elevated heart rate due to heart failure or ischemia, though it may also benefit patients with or without an elevated heart rate who could be helped by having their heart rate reduced, such as patients with high blood pressure due to cardiovascular disease.

In one aspect, a lead with electrodes is implanted in a patient such that the electrodes are in electrical communication with tissue proximate to the right vagus nerve, cardiac plexus, or post-synaptic fibers of the patient. Stimulation circuitry in electrical communication with the lead delivers electrical energy to stimulate the patient through the electrodes. The delivery of electrical stimulus to the right vagus nerve results in a reduction of the patient's normal sinus heart rhythm.

The delivery of electrical energy to the lead may be controlled by control circuitry in accordance with a difference between the actual heart rate of the patient and a maximum “target” heart rate for the patient. A target heart rate, in this regard, may be defined as the highest acceptable heart rate for the patient. The delivery of electrical energy to the lead may also be controlled by control circuitry without a measurement of heart rate but simply timed to occur on a regular basis synthronized with the detection of cardiac contractions.

The lead electrodes may be placed in or near an atrium of the patient's heart. When placed in or near the right atrium, the lead electrodes deliver electrical energy that stimulates parasympathetic nerves in the vicinity of the patient's sinoatrial node, principally the right vagus nerve. The electrodes may also be configured to detect P-waves in the atrium. The delivery of electrical energy to the patient may be synchronized to occur during the time following a P-wave in which cells in the atrium are refractory to stimulation. Heart rate detection circuitry may use the lead electrodes for detecting the patient's actual heart rate from the time interval between the P-waves.

Additional leads with electrodes may also be implanted in the patient's heart. For instance, electrodes of a second lead may be implanted in a ventricle of the patient's heart. Electrodes in a ventricle are capable of detecting R-waves in the heart. Heart rate detection circuitry may determine the patient's actual heart rate from the time interval between the R-waves. In some circumstances, combining R-wave and P-wave detection circuitry into a device may improve the safety of delivering electrical energy to the heart.

In embodiments where a target heart rate is used, the target heart rate for the patient may be automatically adjusted based on current day information obtained from a clock. The current day information may reflect the number of days it has been since the lead was implanted in the patient. In one embodiment, date after implantation (DAI) information is applied to a look up table in memory that is programmed to return a target heart rate for the current DAI.

The target heart rate for the patient may also be automatically adjusted based on current time of day information. For instance, if the current time of day information reflects a time in which the patient is normally sleeping, the target heart rate may be temporarily adjusted downward.

In still another aspect, an activity sensor may be provided to sense the physical activity of the patient. The target heart rate for the patient may be automatically adjusted based on the physical activity sensed in the patient. For instance, if heightened physical activity is sensed, the patient's target heart rate may be temporarily adjusted upward.

The electrical energy may be delivered to the patient in one or more pulses. The amount of stimulation of the patient's vagus nerve (and hence the effect on the patient's heart rate) may be controlled by controlling the voltage, pulse width, or number of pulses delivered to the patient. The voltage, pulse width, or number of pulses may be determined based on the difference between the patient's actual heart rate and the target heart rate.

The invention further provides an electrical lead configured for implantation in a patient's heart. The lead is comprised of a length of electrically conductive material in an insulating substrate and a plurality of electrodes. The electrodes are attached to the insulating substrate and electrically connected to the conductive material. A first electrode is disposed along the length of the insulating substrate and a second electrode is disposed at or near a distal end of the lead. The first and second electrodes are disposed with sufficient distance between them to allow the first electrode to be located in the superior vena cava while the second electrode is located in the atrium near the sinoatrial node when implanted in the heart of the patient. In one embodiment, the electrical lead has a hook-shaped bend in the insulating substrate between the first and second electrodes. In another embodiment, the electrical lead is shaped with one or more helical windings that places the first electrode in the superior vena cava and the second electrode near the sinoatrial node when the lead is implanted in the heart of the patient.

An apparatus constructed in accordance with the invention reduces the heart rate of the patient, with the expectation of improved perfusion of blood through the coronary arteries to the heart muscle.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a pictorial diagram of one exemplary embodiment of an implantable device constructed in accordance with the present invention, with an electrical lead implanted in a patient's heart;

FIG. 1B is a pictorial diagram of another exemplary embodiment of an implantable device constructed in accordance with the present invention, with two electrical leads implanted in a patient's heart;

FIG. 2 is a graph illustrating an example of an atrial electrogram, a detection of P-waves in the atrial electrogram, a ventricular electrogram, and a detection of R-waves in the ventricular electrogram;

FIG. 3 is a block diagram depicting various major components of the device shown in FIG. 1B;

FIG. 4 is a graph illustrating exemplary heart rate reduction curves that set forth target heart rates for the patient over a period of time;

FIG. 5 is a graph illustrating a diurnal variation of a patient's target heart rate, in this case a reduction of the target heart rate during a time in which the patient is normally sleeping;

FIG. 6 is a flow diagram of a method by which the device shown in FIG. 1A or 1B sets a target heart rate for the patient;

FIG. 7 is a flow diagram of another method by which the device shown in FIG. 1A or 1B sets a target heart rate for the patient;

FIG. 8 is a flow diagram of yet another method by which the device shown in FIG. 1A or 1B sets a target heart rate for the patient;

FIG. 9 is a flow diagram illustrating one example of a method of operation for the device shown in FIG. 1B;

FIG. 10 is a graph of a curve illustrating a variance in stimulus voltage according to a difference between an actual heart rate and a target heart rate for a patient;

FIG. 11 is a graph of a curve illustrating a variance in the number of pulses delivered to a patient in accordance with a difference in actual heart rate and target heart rate for the patient;

FIG. 12 is a graph of a curve illustrating a variance in stimulus pulse duration according to a difference between the patient's actual heart rate and target heart rate;

FIG. 13 is a graph illustrating curves showing an increasing heart rate and reduction thereof in accordance with the present invention;

FIG. 14 is a graph of two curves illustrating delivery of stimulus energy to the patient as a function of the patient's heart rate;

FIG. 15 is a graph illustrating a variance in the number of pulses delivered to a patient as a function of the patient's heart rate;

FIG. 16 is a graph illustrating a variance of a stimulus to heart beat ratio as a function of the patient's heart rate; and

FIG. 17 is a pictorial diagram of another exemplary embodiment of an implantable device according to the present invention having a helical electrical lead with a plurality of electrodes in a patient's heart.

### DETAILED DESCRIPTION OF THE PREFERRED EMBODIMENT

To appreciate the present invention, it is helpful to first understand aspects of a patient's nervous system and the interaction of the nervous system with the patient's heart. The parasympathetic nervous system is a part of a patient's overall autonomic nervous system. The parasympathetic nervous system primarily contains cholinergic fibers. Stimulation of a patient's parasympathetic nervous system tends to induce secretion of acetylcholine, increase the tone and contractility of smooth muscle, and to slow the heart rate. The cholinergic nerves to the heart are the right and left vagii.

The right vagus innervates the sinoatrial (SA) node, the atrial muscle, and to a much lesser degree, the atrioventricular (AV) node. The left vagus innervates the AV node, and to a lesser degree, the SA node and atrial muscle. Stimulus of the vagii nerves results in the release of acetylcholine, the amount of which is related to the magnitude of the stimulation. Acetylcholine released by a stimulated right vagus nerve is quickly taken up by the SA node and acts to increase the delay from the current heart beat to the next heart beat. Because the right vagus nerve is known to be distributed primarily in the area of the SA node, electrical stimulation of tissue surrounding the SA node results in stimulation of the right vagus nerve. The present invention takes advantage of the phenomenon that stimulation of the right vagus nerve tends to slow the rate of excitation of the SA node and thereby reduce the patient's heart rate. As used in this patent document, the terms “right vagus nerve” and “vagus nerve” refers to and includes those nerves of the patient that control the delivery of acetylcholine that is taken up by the SA node.

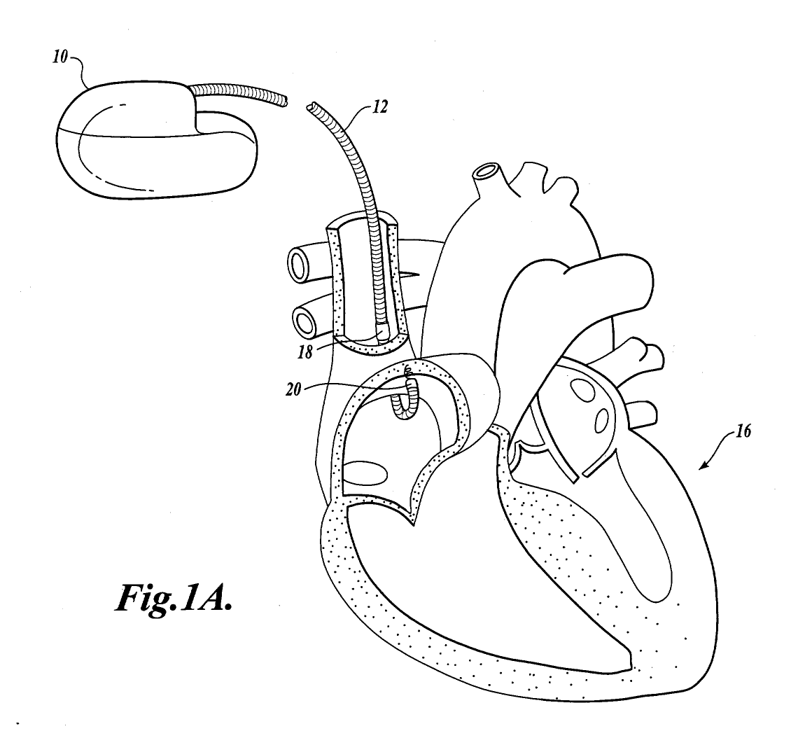

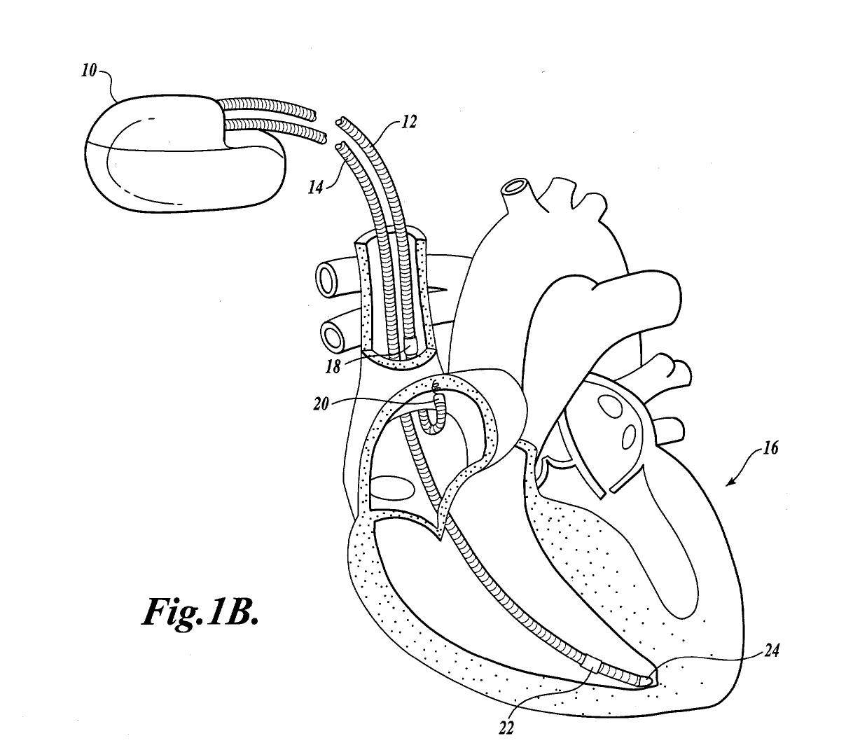

FIG. 1A illustrates one exemplary embodiment of an implantable device 10 constructed in accordance with the present invention. In the embodiment shown, the device 10 is attached to a catheter containing an electrical lead 12 that has been implanted in a patient's heart 16. The electrical lead 12, as illustrated, has a ring electrode along the length of the lead and a tip electrode at or near the end of the lead. In particular, the electrical lead 12 has a ring electrode 18 positioned near the junction of the superior vena cava (SVC) and tip electrode 20 in the right atrium of the heart 16.

The electrodes 18 and 20, as illustrated, are positioned to sense electrical signals in the atrium of the heart 16 that reflect atrial activity, which signals are relayed to the device 10 for processing by control circuitry in the device 10. The electrodes 18 and 20 are also positioned to stimulate the portion of tissue proximate to the patient's right vagus nerve and SA node. Tissue is proximate to the right vagus nerve when stimulation of the tissue results in release of acetylcholine from the vagus nerve. The acetylcholine is taken up by the SA node.

Furthermore, the tip electrode 20 may include a clamp or helical screw, as shown in FIG. 1A, to better secure the electrode to the atrial tissue. As noted earlier, stimulation of the right vagus nerve, in accordance with the present invention, is designed to release acetylcholine and increase parasympathetic tone in order to slowly reduce the normal rate of excitation of the SA node.

The electrodes 18 and 20 may be comprised of electrodes commonly used in cardiac sensing and pacing. However, the atrial lead 12 is different than prior art atrial pacing leads in that the spacing between the electrodes 18 and 20 may be longer than 1 to 2 cm, as is common in pacing leads. This spacing is useful in the present invention to capture a wider area of cardiac tissue, which improves the ability to ensure the right vagus nerve fibers are located in the current path between the electrodes 18 and 20. The atrial lead 12 is also different than prior art pacing leads in that the atrial lead 12 may include a relatively sharp preshaped bend between the electrodes 18 and 20. The sharp bend enables the atrial lead 12 to effectively wrap around the junction of the SVC and right atrium, and surround the tissue where the right vagus nerve and SA node are located.

FIG. 1B illustrates another exemplary embodiment of implantable device 10 that includes the electrical lead 12 shown in FIG. 1A, and additionally a catheter containing an electrical lead 14 that has been implanted in a patient's heart 16. The electrical lead 14 includes a ring electrode 22 and tip electrode 24 positioned within the right ventricle of the heart 16. The ring electrode 22 and tip electrode 24 are positioned to sense electrical signals that reflect ventricular activity, which signals are relayed to the device 10 for processing by control circuitry in the device 10. The ventricular lead 14 may be constructed using a bipolar RV pacing lead which is well known in the pacing art. When plotted, the electrical signals from the atrium (sensed through lead 12) produce an atrial electrogram. The electrical signals detected in the ventricle (sensed through lead 14) produce a ventricular electrogram.

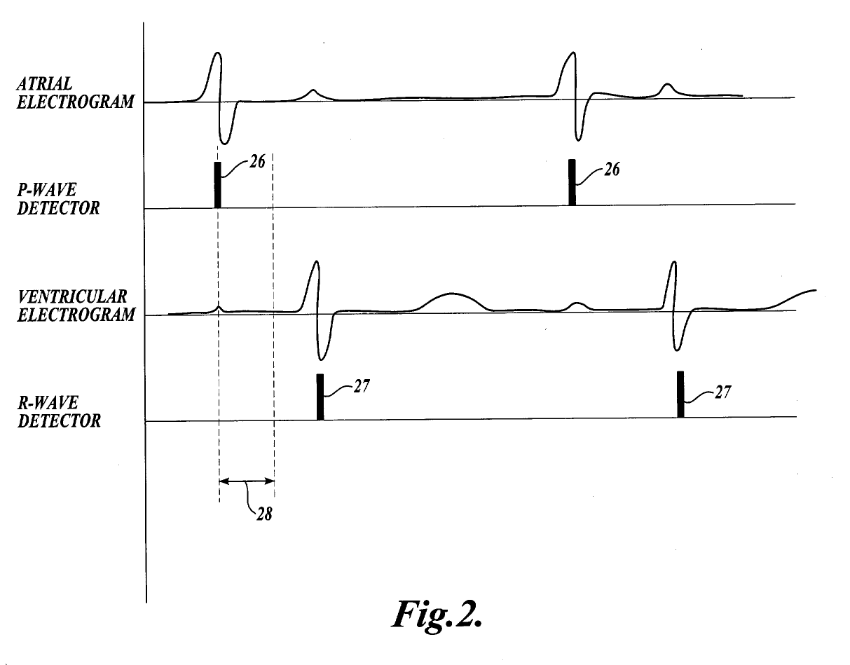

FIG. 2 illustrates an example of an atrial electrogram and ventricular electrogram for a patient with a normal sinus rhythm. The peak values in the atrial electrogram reflect electrical activity that results from contraction of the atrium. This peak in atrial electrical activity is commonly referred to as a P-wave. Subsequent to contraction of the atria, electrical activity in the purkinje system causes the ventricles to contract, pushing blood into the patient's peripheral circulation system. The peak electrical activity in the ventricular electrogram in FIG. 2 is commonly referred to as an R-wave. Some embodiments of the invention detect only P-waves; other embodiments include detection of R-waves. In any event, detection of R-waves is not necessary to practice the present invention.

In some implementations of the device 10, it may be advantageous to further include components that monitor the patient's blood pressure and provide the blood pressure information to the control circuitry in the device 10 that controls the operation of the device. One example of a suitable blood pressure sensor measures impedance and may be applied to the surface of the subclavian or other convenient artery. Another example of a suitable sensor is a piezoelectric device attached to an artery that generates a voltage pulse reflective of the patient's blood pressure. As the device 10 slowly lowers the normal sinus rate of the patient, the blood pressure is monitored by the control circuitry to ensure it remains within an acceptable range. For instance, the control circuitry may include a memory with programmed blood pressure limits in the form of a look up table. If the detected blood pressure drops beyond limits designated in the look up table, the control circuitry of the device 10 may be configured to produce a control signal that causes the amount of stimulation being delivered to the patient to be reduced. By lowering the amount of parasympathetic stimulation, the effect on the patient's heart rate should be reduced, thereby allowing the heart to maintain blood pressure at an acceptable level. As the patient's blood pressure stabilizes, the amount of vagal stimulation may be increased back to previous levels.

As noted earlier, the ring electrode 18 and tip electrode 20 are placed in close proximity to the atrial tissue next to and surrounding the right vagus nerve and ganglionic tissue. The stimulation of the right vagus nerve in this embodiment preferably occurs during the refractory period 28 (FIG. 2) following the P-wave in the atrium. For human patients, the atrial refractory period 28 is generally considered to be about 100 ms in duration. During the refractory period 28, the cells in the atrial muscle tissue are not susceptible to direct stimulation. However, the vagus nerve fibers can be stimulated during this time to release acetylcholine that is taken up by the SA node, causing a longer delay by the SA node before initiating the next P-wave. The amount of delay to the next atrial contraction is roughly proportional to the amount of energy delivered to the right vagus nerve in the stimulus.

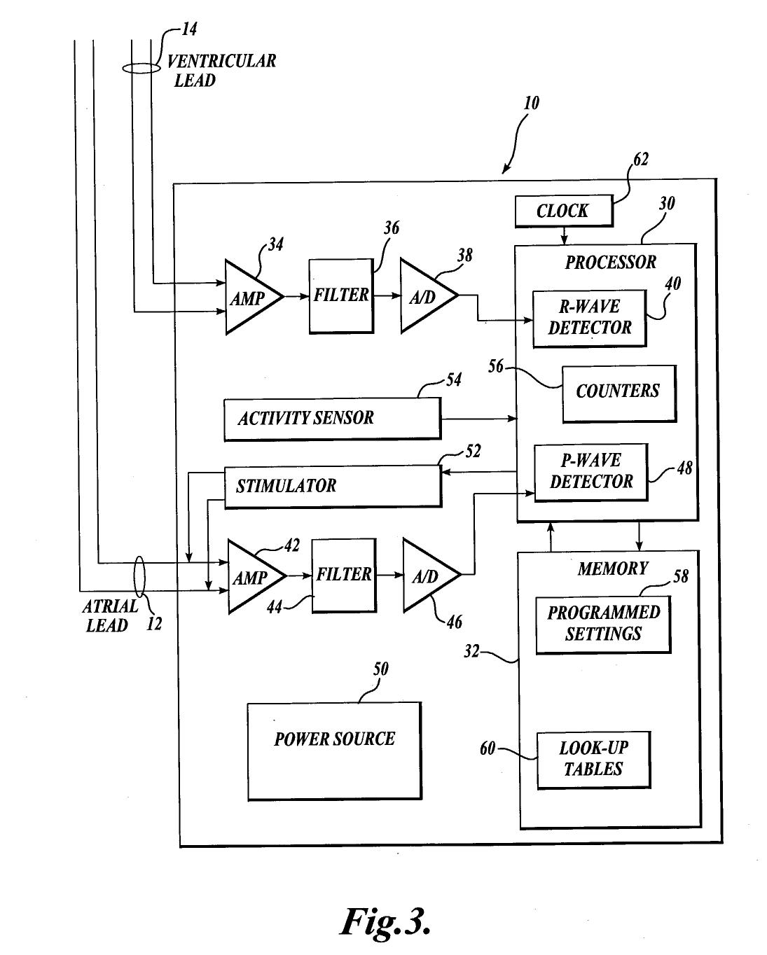

As shown in FIG. 3, a P-wave detector 48 in the device 10 monitors the atrial electrogram for a peak value that represents the P-wave in the patient's heat. When a P-wave is detected, the P-wave detector produces a logical output 26 (FIG. 2) that marks the detection of a P-wave. In similar fashion, embodiments of the invention that include R-wave detection have an R-wave detector 40 that monitors the patient's ventricular electrogram for a peak value indicative of an R-wave. When an R-wave is detected, the R-wave detector produces a logical output 27 that marks the detection of an R-wave in the patient's heart. The construction and function of P-wave detectors and R-wave detectors are well known in the art. Further information concerning the construction of the device 10, including P-wave and R-wave detectors, is provided below in regard to FIG. 3.

The amount of energy in the stimulation delivered to the patient may be controlled by varying the voltage, duration, and/or frequency of the electrical pulses providing the stimulation. A higher voltage, longer duration, or higher number of pulses, with other factors kept equal, leads to a greater amount of energy being delivered in each stimulus, thus leading to an increased time interval to the next heart beat after each stimulus. Depending on patient circumstances and placement of the electrical leads, a pulse train longer than the atrial refractory period 28 should be avoided as it may directly stimulate an atrial contraction, thus speeding the heart rate rather than slowing it.

FIG. 3 is a block diagram illustrating various major components of one embodiment of the device 10 shown in FIG. 1B. As described above, the device 10 includes control circuitry that controls the operation of the device. In the embodiment shown in FIG. 3, the control circuitry of the device 10 includes processor circuitry 30 in communication with a memory 32. The memory 32, which may be formed of any type of memory components, such as RAM, ROM, flash memory etc., may include programmed instructions carried out by the processor circuitry 30. In addition to or in place of programmed instructions, the processor circuitry 30 may also operate in accordance with hardwired circuitry in or connected to the processor circuitry 30.

As shown in FIG. 1B, the atrial lead 12 includes two electrodes 18 and 20 positioned near or within the right atrium of the patient. Electrical activity sensed by these electrodes is communicated via the electrical lead 12 to one or more amplifiers 42 (FIG. 3). The amplified signals are filtered in one or more filters 44, which may be used to attenuate noise and/or other signal contaminants, for example. The one or more filters 44 may also be used to emphasize portions of the signals that are of particular interest. The filtered signals are then converted from analog to digital form in an analog-to-digital (A/D) converter 46. The signal information from the A/D converter 46 is delivered to the P-wave detector 48. In FIG. 3, the P-wave detector circuitry 48 is shown integrated with the processor circuitry 30. However, in other suitable embodiments, the P-wave detector 48 may be implemented by separate circuitry in communication with the processor circuitry 30.

The amplifier(s) 42, filter(s) 44 and A/D converter(s) 46 used in the device 10 may be constructed of conventional off-the-shelf components. Similarly, the amplifier(s) (as) 34, filter(s) 36, and AID converter(s) 38 (discussed below) may also be constructed of conventional components. The selection and implementation of appropriate amplifiers, filters, and A/D converters is well within the ability of one having ordinary skill in the art of implantable devices for signal processing and cardiac therapy.

Electrical activity sensed by electrodes 22 and 24 in the right ventricle (FIG. 1B) are delivered to the device 10 via the ventricular lead 14. As shown in FIG. 3, the electrical signals in the ventricular lead 14 are amplified by one or more amplifiers 34 and filtered by one or more filters 36. The amplified and filtered signals are then converted from analog to digital form in an A/D converter 38. The resulting digitized signals are then delivered to an R-wave detector 40 operating within or connected to the processor circuitry 30. Embodiments of the invention that do not include a ventricular lead 14, such as the embodiment shown in FIG. 1A, may exclude the amplifier 34, filter 36, A/D converter 38, and R-wave detector 40 as shown in FIG. 3.

A power source 50 in the device 10, typically a long-life battery such as a lithium battery, provides electrical power to the sensors and circuitry in the device 10. A clock 62 in communication with the processor circuitry 30 keeps track of the current date and time, for purposes explained below. The clock 62 may also provide timing information as needed to the processor circuitry 30. Conventional components for implementing the clock 62 are well known and commercially available to those having ordinary skill in the art.

The device 10, as illustrated in FIG. 3, includes further components, the function and operation of which are described below in more detail. These additional components include a stimulator 52 connected to the electrodes 18 and 20 on the atrial lead 12, an activity sensor 54 that senses physical activity of the patient, counters 56 integrated within or connected to the processor circuitry 30 for monitoring the patient's heart rate and delivery of therapy to the patient, programmed settings 58 in the memory 32 that include various target values and limits used in the operation of the device 10, and lookup tables 60 in the memory 32 used primarily to set the programmed settings 58. Those of ordinary skill in the art will recognize that FIG. 3 represents only one exemplary embodiment of the device 10. Other suitable embodiments of a device constructed according to the principles of the invention may include more or fewer components than those illustrated in FIG. 3. For example, the embodiment shown in FIG. 1A may include fewer components than the embodiment in FIG. 1B. Moreover, components (such as the stimulator 52 and processor circuitry 30) shown in FIG. 3 may be integrated or kept separate, as desired. The components shown in FIG. 3 are intended to illustrate the operation of at least one preferred embodiment of the invention, and are not intended to limit the scope of the invention. The features of the invention may also be integrated into other commercially available products, such as a conventional DDDR pacemaker, an ICD, and/or a biventricular pacemaker.

The device 10 delivers stimulation to the right vagus nerve to reduce the patient's heart rate. The stimulator 52, preferably controlled by the processor circuitry 30, delivers electrical pulses to the right vagus nerve via the electrodes 18 and 20 on the atrial lead 12. The stimulator 52 may be comprised of any type of circuitry capable of delivering electrical energy to the atrial lead 12. For example, the stimulator 52 may be a programmable pulse generator that receives command input from the processor circuitry 30 that directs the voltage, pulse width, and/or number of pulses to be delivered from the stimulator 52 at each stimulus cycle. Alternatively, the stimulator 52 may be an electrode driver circuitry capable of delivering an electrical signal at a specified voltage. In that circumstance, the processor circuitry 30 controls the switching (on/off) of the driver circuit, as well as the voltage, to deliver the electrical pulses in the stimulus. Other suitable constructions for delivering electrical pulses may be used. Programmable pulse generators and electrode driver circuits as noted above are well known to those of ordinary skill in the art.

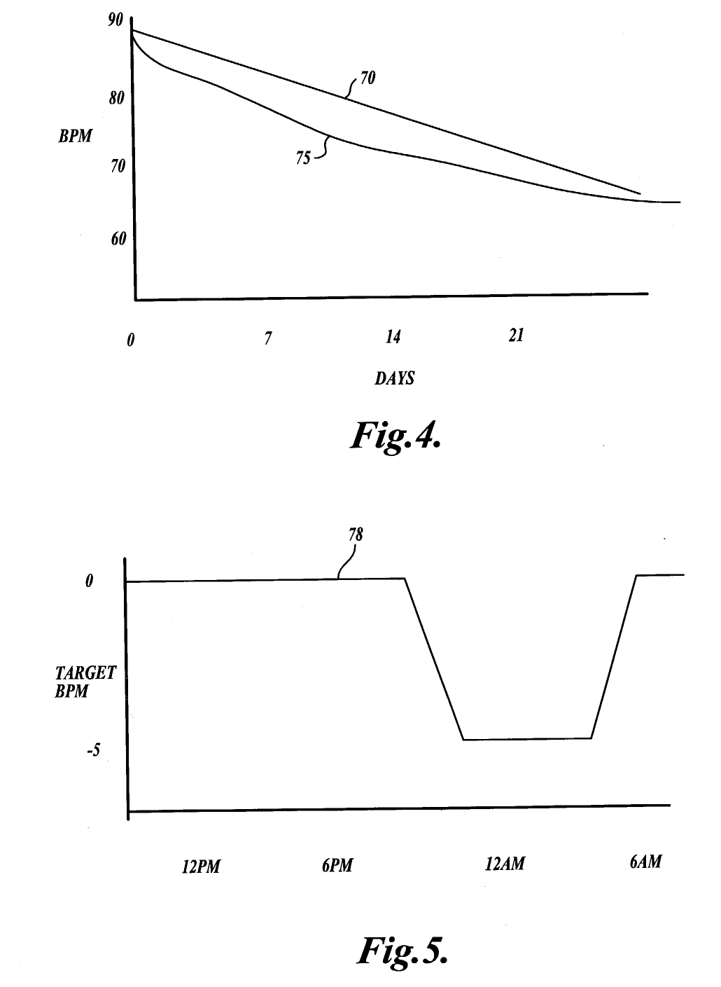

For embodiments of the invention configured to drive a patient's heart rate downward to a maximum target rate, the target heart rate is preferably set by a physician. FIG. 4 illustrates heart rate reduction curves that may be programmed in the device 10 by a physician, as discussed further below. The curves depicted in FIG. 4 extend over a time period of three weeks measured in days after implantation (DAI) of the device 10. In one implementation, the reduction in heart rate over time may be approximately linear, as shown by the curve 70 in FIG. 4. In this example, the patient, at Day 0, has a target heart rate of 90 beats per minute (bpm). According to curve 70, over the following three weeks, the patient's heart rate is expected to drop by approximately 5 bpm over each seven-day period, for an overall reduction of 15 bpm at the end of three weeks. The heart rate reduction may continue for additional weeks or months to reach a final desired maximum heart rate.

The reduction in heart rate may also be set to follow a non-linear curve. In some circumstances, a physician may determine that a non-linear reduction will better serve the patient's health. For example, it may be determined that the patient is better served by a faster reduction in heart rate over an initial period of time, with a slower reduction of heart rate over a later period of time. See, e.g., the reduction curve 75 shown in FIG. 4. In other circumstances, it may be determined better for the patient to have a slower reduction in heart rate initially, with an increased reduction in heart rate in a later period of time. It is also contemplated that the reduction curve can be continuous (as shown) with changes in the target rate throughout each day, or it may be stepped with as few as one discrete change in target rate each day or each few days.

In any event, it is anticipated that the reduction in heart rate will not be so rapid as to significantly affect the patient's sense of being. For example, as noted earlier, a reduction in heart rate that is too rapid may lead to a rapid reduction of blood pressure, resulting in patient fatigue or fainting. An implementation of the device 10 that includes blood pressure monitoring circuitry, as discussed above, may assist in maintaining the patient's blood pressure within an acceptable range.

In circumstances where the patient is experiencing high blood pressure, e.g. from cardiovascular disease, a reduction in blood pressure may-be desired. In accordance with the present invention, a patient's heart rate may be reduced in a manner that results in a desired reduction of blood pressure. Provided that the patient's blood pressure remains within a medically-acceptable range, the amount of electrical stimulation delivered to the patient may be varied based on the amount of blood pressure reduction that is desired. In some embodiments, a maximum target blood pressure may be programmed in the device, much like embodiments discussed herein that use a maximum target heart rate. The difference between the patient's current blood pressure and the target blood pressure may be used to control the delivery of electrical stimulus to the patient.

A physician preferably determines a final maximum target heart rate and a heart rate reduction curve for a patient after evaluating the patient's condition. The device 10 may be configured with the target rate and heart rate reduction curve information prior to or after implantation of the device 10 in the patient. If programming occurs after implantation, the programming may be accomplished via wireless communication with the device 10. The construction of appropriate RF transmitters and receivers for telemetry of data into and out of the device 10 implanted in the patient is well known. One having ordinary skill in the art may construct an appropriate transmitter and receiver for wireless programming of the device 10 using conventional components. The final target rate and heart rate reduction curve information may be stored within the lookup tables 60 shown in FIG. 3.

A physician may also determine it advantageous to have the device 10 automatically adjust the target heart rate according to the physical activity of the patient. In one aspect, a device 10 constructed according to the invention may include a diurnal variation in the target rate that reflects the time in which the patient is sleeping. For example, FIG. 5 illustrates a target rate reduction curve 78 for a patient who sleeps during normal night hours. As shown in FIG. 5, during the daytime, there is no reduction of the target heart rate set in the programmed settings 58 of the device 10. Commencing at about 9:00 p.m., the target rate is lowered until it reaches a designated target rate reduction. The example in FIG. 5 shows a target rate reduction of 5 bpm, though the designated reduction of target rate may be more or less, as desired. During the normal night hours, the target heart rate is thus 5 bpm lower than it is during the day. Commencing at approximately 5:00 a.m., the target rate reduction is phased out, and the device returns to normal daytime operation with no reduction in the patient's target rate.

The target rate reduction curve 78 shown in FIG. 5 is exemplary only, and does not reflect any particular limitations of the invention. For other patients, the times of day at which a reduction occurs may be different. The variation in target rate may also be more than diurnal, and include multiple times when the patient is expected to be inactive.

A device 10 constructed according to the invention may also include an activity sensor 54 that monitors the patient for elevated physical activity, such as exercise. The activity sensor 54 may also respond to parameters such as blood temperature, respiratory rate, body motion, etc., as are well known in the art. See, e.g., Geddes et al., “The Exercise-Responsive Cardiac Pacemaker,” IEEE Transactions on Biomedical Engineering, 31(12) (December 1984), incorporated by reference herein. In response to detected physical activity, the target heart rate of the patient may be temporarily adjusted upward for the period of time in which the increased physical activity is detected. The increase in target heart rate may also be programmed to follow a target rate enhancement curve that permits the patient to have an adequate heart rate to sustain the physical activity while the activity is occurring. When the heightened physical activity is done, the device returns to the normal target heart rate programmed for the patient, either immediately or according to the programmed target rate enhancement curve.

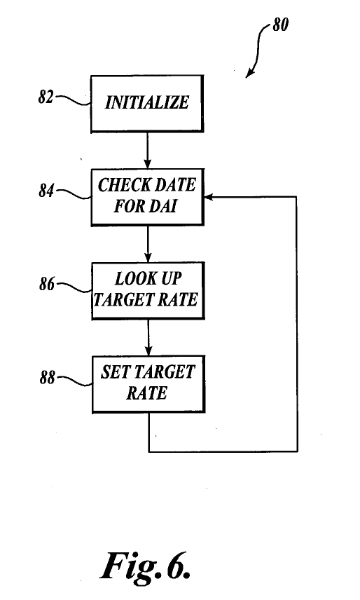

FIGS. 6-8 illustrate various methods by which the device 10 may set a maximum “target” heart rate for a patient during normal device operation. The method 80 shown in FIG. 6 commences at block 82 in which the device 10 is initialized. The initialization process in block 82 includes all aspects of setting up the device for normal operation, including programming the final target rate, the heart rate reduction curve, and the target rate reduction enhancement curve information in the look-up table 60 as discussed earlier.

The clock 62 (FIG. 3) keeps track of current day and time information for the device 10. At block 84, the device 10 checks the current date and time information to determine the number of days it has been since the device was implanted. At block 86, the date after implantation (DAI) information is applied to the lookup table 60 to determine the target heart rate for the current day. For example, as shown in FIG. 4, the heart rate reduction curve 70 for Day 7 reflects a target rate of 85 bpm. For Day 14, the target rate is 80 bpm, etc. The determined target heart rate is returned from the lookup table 60 and is set in the programmed settings 58 (FIG. 3), as shown at block 88, to control the ongoing operation of the device 10 during that day. The method 80 returns to block 84 and repeats the entire process on a periodic basis to insure that the target heart rate set in the programmed settings 58 remains current.

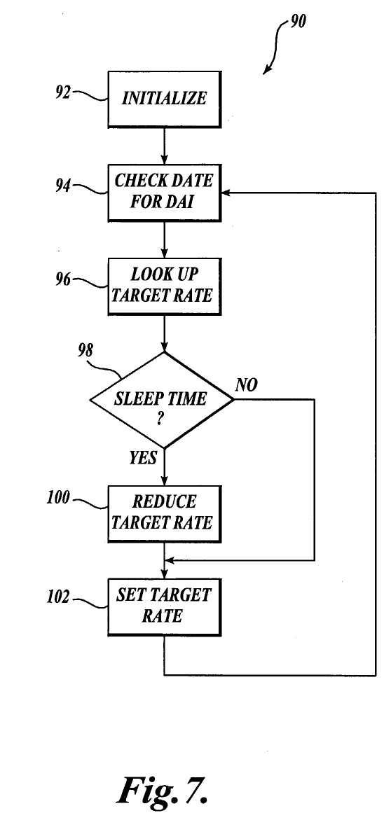

FIG. 7 illustrates an alternative method 90 for setting the patient's target heart rate in the programmed settings 58. The initial tasks in the method 90 are similar to those in the method 80, including initialization in block 92, checking the current date for DAI information in block 94, and looking up the target heart rate for the current day in block 96.

Once the target rate is determined in block 96, the method 90 checks the current time to determine whether it is during the patient's scheduled sleep time. If, at decision block 98, the current time is during the patient's sleep time, the target rate is reduced (block 100) according to a programmed target rate reduction curve, such as the curve 78 shown in FIG. 5. At block 102, the reduced target is then set in the programmed settings 58 to control the ongoing operation of the device 10.

Returning to decision block 98, if the current time is not during the patient's sleep time, the target rate determined at block 96 is set in the programmed settings 58 to control the ongoing operation of the device 10. In either case, the method 90 then returns to block 94 and repeats the process on a periodic basis to insure that the target rate set in the programmed settings 58 remains current.

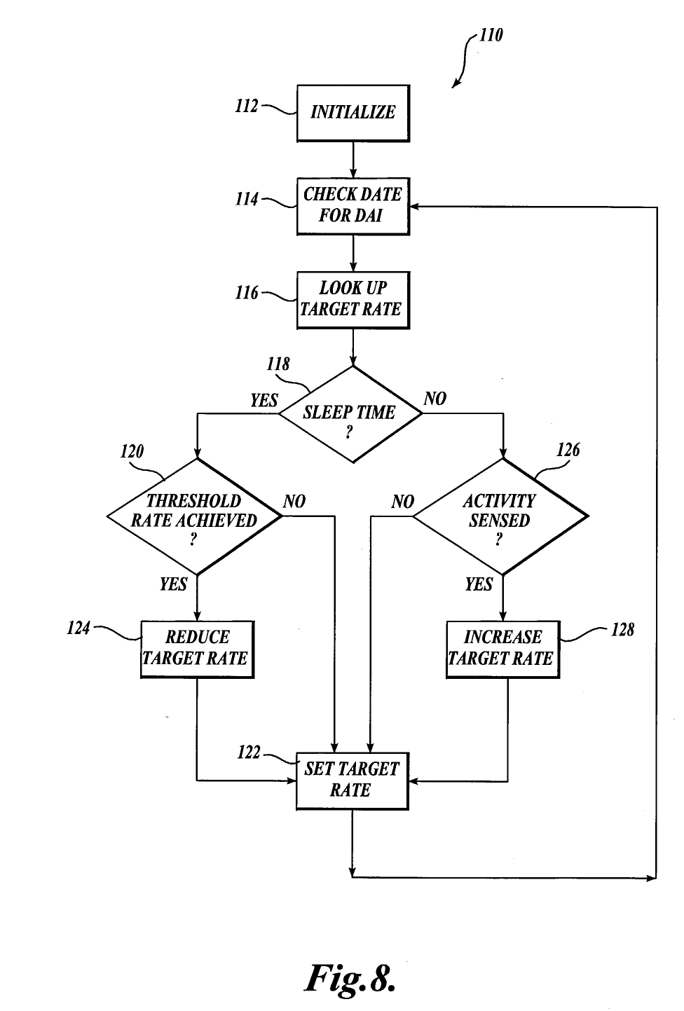

FIG. 8 illustrates yet another method 110 for setting the patient's target heart rate and includes additional alternative features. The initial tasks performed in the method 110 are similar to those performed in the method 80 in FIG. 6, including initialization in block 112, checking the current date for DAI information in block 114, and determining the target rate for the patient in block 116 based on the DAI information.

At decision block 118, the method 110 determines whether the current time is during the patient's scheduled sleep time. If the current time is during the sleep time, the method 110 progresses to a decision block 120 where it determines whether a minimum reduction in heart rate has been achieved for the patient. For example, a physician may determine that a diurnal variation in the patient's target rate is not appropriate until the patient's heart rate has been reduced to 80 bpm. If a heart rate of 80 bpm has not yet been achieved (in this example), the method 110 progresses from decision block 120 to block 122 at which the target rate determined in block 116 is set in the programmed settings 58. If, at decision block 120, the threshold rate (here, 80 bpm) has been achieved, the device 10 reduces the patient's target rate (block 124) according to a target rate reduction curve, such as the curve 78 shown in FIG. 5. The reduced target rate is then entered into the programmed settings 58, as shown at block 122.

Returning to the decision block 118, if the current time is not during the patient's sleep time, the method 110 progresses to a decision block 126 at which the device 10 determines whether the patient is experiencing heightened physical activity. If heightened physical activity is sensed, the device 10 may temporarily increase the patient's target rate (block 128) according to a target rate enhancement curve to accommodate the patient's physical activity. The increased target rate is then set in the programmed settings 58, as shown at block 122. If heightened physical activity is not sensed at block 126, the device 10 sets the target rate determined at block 116 in the programmed settings 58.

In any event, after the target rate is set at block 122, the method 110 returns to block 114 and repeats the process on a periodic basis to insure that the programmed target rate remains current and reflects the diurnal variation or physical activity of the patient.

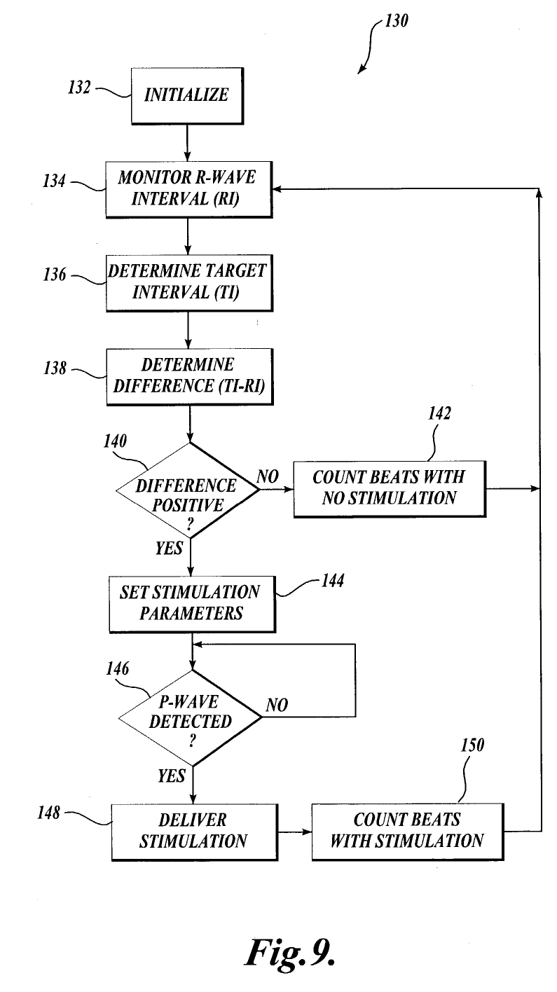

Before discussing specific aspects of the amount of stimulation delivered by the device 10, it is useful to first observe the overall operation of at least one embodiment of the device 10. FIG. 9 illustrates one example of an operational method 130 for the device 10. At block 132, the device 10 undergoes initialization processes, including those processes that set up the device 10 for ongoing operation. The initialization in block 132 includes the process of setting up the target rate, as shown by the methods in FIGS. 6-8. Once the initialization processes are complete, the method 130 commences a repeated routine in which the device 10 monitors the patient's ventricular electrogram for R-waves, as indicated at block 134. The patient's heart rate can be calculated from the frequency of detected R-waves. In the embodiment shown in FIG. 9, the method 130 calculates the time interval “RI” between detected R-waves.

Alternatively, the patient's heart rate can be calculated from the frequency of detected P-waves in the atrium. In that regard, the method 130 may calculate at block 134 the time interval between detected P-waves. This may be particularly useful for embodiments of the invention having a single lead in the atrium and not having a lead in a ventricle of the heart. In a normal sinus rhythm, the P-wave interval and R-wave interval are approximately the same. Accordingly, discussion of R-wave intervals herein is equally applicable to detection and use of P-wave intervals.

At block 136, the device 10 uses the target heart rate set in the programmed settings 58 to determine the target time interval “TI” between R-waves. The difference between the target interval TI and the actual R-wave interval RI is calculated at block 138.

To avoid skewed or erroneous information obtained for any particular R-wave interval detected in the patient, the device 10 may calculate several R-wave intervals and determine an average for the time interval RI. The device 10 may exclude specific R-wave interval calculations that reflect obvious errors in view of the average time interval RI.

Once the difference between the target interval TI and the actual interval RI is calculated, the device 10 determines whether the difference is positive, as shown at decision block 140. A positive difference indicates that the patient's actual heart rate is higher than the target rate, thus indicating that stimulation of the patient's right vagus nerve in accordance with the invention is warranted. If the determined difference is not positive (thus indicating that the patient's actual heart rate is lower than the targeted rate), stimulation is not delivered. Instead, a counter (such as counter 56 shown in FIG. 3) that monitors heart beats without stimulation is incremented, as indicated at block 142.

If the TI-RI difference is positive, the device 10 sets the stimulation parameters at block 144. Additional detail on the manner in which the stimulation parameters are set is provided below. Progressing to decision block 146, the method 130 determines whether a P-wave has been detected in the patient's atrial electrogram. The P-wave detection in block 146 is repeated until a P-wave is detected. At block 148, the electrical stimulation is then delivered to the patient. A counter (such as counter 56 shown in FIG. 3) that monitors heart beats with stimulation is incremented at block 150, and the method 130 returns to block 134 and is repeated.

To deliver the stimulus to the right vagus nerve during the atrial refractory time 28, the stimulus is preferably synchronized with the P-wave. As noted earlier, the atrial refractory period is generally considered to be about 100 ms in duration. Therefore, the electrical stimulation of the vagus nerve should occur during a 100 ms time period commencing with or shortly after a P-wave is detected. The stimulus may be a single pulse, a burst of pulses, a steady train of pulses, etc., during the atrial refractory period.

It should be noted that in implementations where the electrodes for stimulation of the right vagus nerve are placed away from the heart, such as within the right pulmonary artery or in the superior vena cava, the need to synchronize the stimulus with the P-wave diminishes. In such circumstances, there is a lesser concern of direct stimulation of the atrial muscle cells. Nevertheless, it may still be advantageous to synchronize the stimulus with the P-wave of the atrium to maintain the integrity of the stimulus and response.

It should also be noted that the present invention does not require delivery of an electrical stimulus after every heart beat (when stimulation is warranted). The electrical stimulation of the right vagus nerve may be scheduled to occur every few heart beats, for example. Separating the stimuli by several heart beats may provide certain advantages. For instance, it provides the device 10 with some flexibility to increase the stimulus by reducing the number of non-stimulated heart beats between each stimulus delivery. The device 10 may also increase the number of non-stimulated heart beats between stimulus delivery as the patient's heart rate nears the target heart rate.

As discussed above, the counter 56 (FIG. 3) counts the number of heart beats where a stimulus was delivered, as well as the number of heart beats without stimulus delivery. This information may be stored in the memory 32 and communicated by wireless transmission to a receiver (not shown) outside the patient for physician review. By observing the number of heart beats with and without stimulation, as well as the progress of the patient towards the target heart rate, the physician can better evaluate the efficacy of the device 10 as currently programmed. It may be that for certain patients, adjustments in the programming of the device 10 is needed to properly stimulate the patient toward the target heart rate. Nevertheless, counting the number of heart beats with and/or without vagal stimulation is optional and not necessary to practicing the present invention.

The programming of the device 10 may be adjusted by the physician, or it may be done automatically by the device 10. For the latter, the device 10 is configured to provide feedback information to its control circuitry as to the patient's progress toward the target heart rate. If the device 10 observes that patient's actual heart rate is not progressing toward the target rate, the device may automatically increase the energy in each stimulus. The amount and frequency that the device 10 automatically increases the energy may be constrained by limits set in the device to ensure that the stimulation does not become excessive.

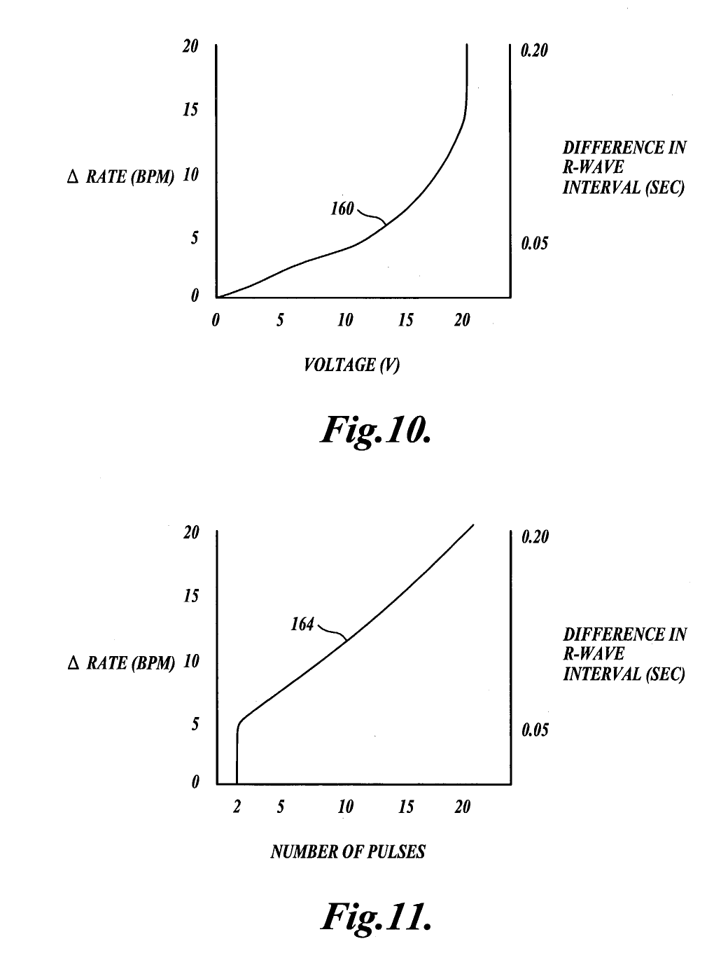

The amount of effect the stimulation has on slowing a patient's heart rate is proportional to the amount of energy in the stimulation delivered to the right vagus nerve. The energy delivered in the stimulation may thus be varied to adjust the rate at which the patient's heart rate is reduced. In one aspect, a device constructed according to the present invention, such as device 10, may vary the voltage of the stimulus to vary the energy delivered by the stimulus. As shown in FIG. 10, the stimulation voltage may vary according to a curve, such as curve 160. The greater the difference between the target heart rate and the actual heart rate (or alternatively, the difference in R-wave interval), the greater the voltage of the electrical stimulus that is delivered to the patient. A maximum voltage that can be delivered to the patient may be set in the device or may be constrained by limitations of the components in the device. In the example shown in FIG. 10, a maximum voltage of 20 volts is provided.

The amount of energy delivered to the patient may also be varied by delivering a number of electrical pulses to the patient. The number of pulses is varied depending on the difference between the patient's actual heart rate and the target rate. For example, as shown by the curve 164 in FIG. 11, the greater the difference in heart rate (or difference in R-wave interval), the greater the number of pulses that are delivered to the patient in each period of stimulation. Per the example shown in FIG. 11, it is anticipated that the voltage of each pulse is constant (e.g., 1-20 volts as set in the device). The pulse width of each pulse is also constant (e.g., 0.1-0.5 ms as set in the device). As the electrical stimulus is synchronized with the P-wave, the number of pulses determined by the curve 164 may be spread evenly throughout the atrial refractory period, or alternatively may be set at a frequency to all occur within a portion of the refractory period, such as the initial part of the refractory period.

As to frequency, a device constructed in accordance with the present invention may implement an equation as follows:

$${F}_{stim} = \frac{Actual Rate}{Target Rate} \times K + I$$

In the foregoing equation, K is a variable constant that can be increased if the actual rate exceeds the target rate and the amount of vagal stimulation is ineffective in lowering the actual rate toward the target rate. The variable constant I represents a starting frequency.

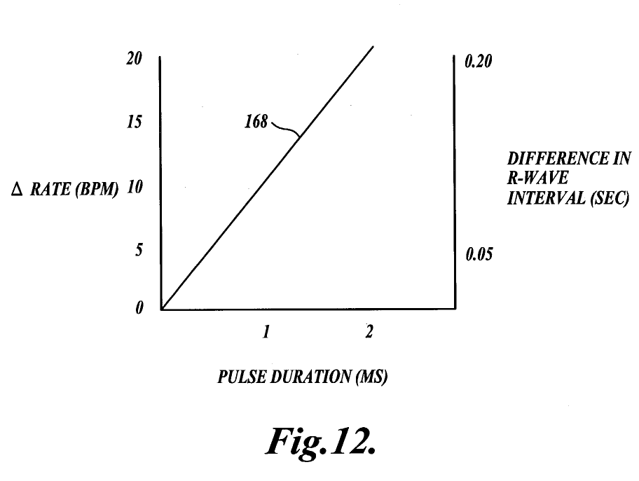

FIG. 12 illustrates another example of an embodiment of the invention that varies the duration of pulses in a pulse train to vary the amount of energy delivered in the stimulus. The curve 168 shown in FIG. 12 is approximately linear and demonstrates an increase in pulse duration commensurate with the difference between the patient's actual heart rate and the patient's target rate (or difference in actual to target R-wave interval). However, linear increase in pulse duration is not necessary and in fact a non-linear increase may be warranted. For the example shown in FIG. 12, it is anticipated that the voltage and number of pulses in the stimulus remain constant, though the number of pulses may be limited by the overall duration of the patient's atrial refractory period.

Because the present invention is directed to lowering the heart rate of a patient, it may be advantageous to further include circuitry for delivering pacing pulses to the patient. Unlike the present invention, a pacing pulse is designed to initiate a heart beat to maintain a desired minimum heart rate. Including pacing circuitry in the device 10 would act as a safety net in case the patient's heart rate dropped unreasonably below the set target rate for the patient. Cardiac pacing is a well-developed field of technology. The components and construction of pacing circuitry for use in the device 10 is within the ability of one of having ordinary skill in pacing art, and may use existing components of the device 10, such as the processor, pulse detection, and stimulator circuitry. For example, components of a conventional DDDR pacemaker, ICD, or biventricular pacemaker may be incorporated into the device 10 (or vice versa).

While various preferred embodiments of the invention have been illustrated and described, it will be appreciated that insignificant changes can be made therein without departing from the spirit and scope of the invention. For instance, the method of operation shown in FIG. 9 is only exemplary of one embodiment of the invention. Another embodiment of the invention (e.g., as shown in FIG. 1A) may monitor a P-wave interval instead of R-wave interval as shown in block 134 of FIG. 9, and achieve the same results. Further embodiments of the invention that achieve the same advantages, including a reduction of a patient's normal sinus heart rate to allow improved perfusion through the coronary arteries, are within the ability of one having ordinary skill in the art.

Other embodiments of the invention may sense the heart rate of the patient differently than described above. Rather than sensing R-waves or P-waves through leads implanted in the heart, the device 10 may include pulse detection circuitry that uses electrodes placed elsewhere in the patient's body. For instance, the pulse detection circuitry may use electrodes to sense impedance changes in an artery or may use piezoelectric sensors on an artery to sense pressure changes resulting from a cardiac pulse. See, e.g., Konrad et al., “A New Implantable Arterial Pulse Sensor for Detection of Ventricular Fibrillation,” Medical Instrumentation, 22(6):304-311 (December 1988) for an arterial pulse sensor, incorporated by reference herein.

Embodiments of the invention discussed above deliver electrical stimuli based on the difference between the patient's actual heart rate and a maximum target heart rate for the patient. Nevertheless, use of a target heart rate for the patient is not necessary to practicing the present invention. Other embodiments of the invention may deliver electrical stimuli in an amount that is predetermined or is based on patient conditions, such as the frequency of cardiac contractions in the patient (i.e., heart rate).

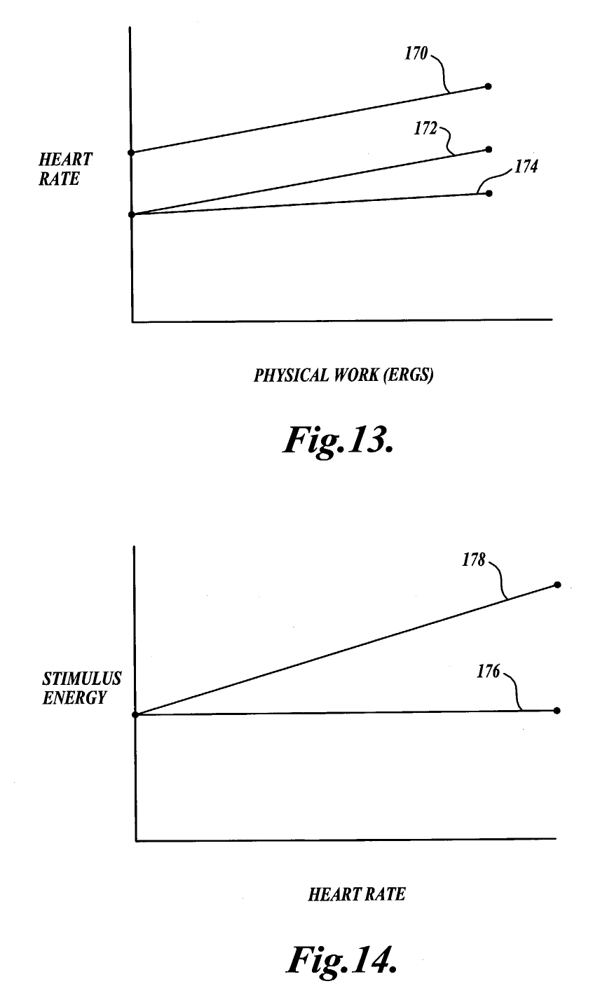

A patient's heart rate typically increases as the patient exerts increased physical work, as reflected by the upper curve 170 in FIG. 13. Embodiments of the invention that deliver a predetermined amount of electrical stimuli to the patient regardless of the patient's heart rate will reduce the patient's heart rate, as shown by the middle curve 172 in FIG. 13. However, the slope of the curve 172 is the same as the curve 170 because the amount of stimulus delivered to the patient does not vary with the heart rate resulting from the amount of physical work being performed by the patient. An embodiment of the invention of this type is also reflected by the curve 176 in FIG. 14. FIG. 14 illustrates two curves showing the amount of energy in electrical stimulus to the patient as a function of the patient's heart rate. The lower curve 176 is constant, regardless of the patient's heart rate.

An embodiment of the invention that increases the amount of stimulus energy as a function of the patient's heart rate is reflected by the upper curve 178 in FIG. 14. As the patient's heart rate increases, the amount of energy delivered to the patient in each stimulus also increases. As noted earlier, the amount by which a patient's heart rate is reduced is proportional to the amount of energy delivered to the patient's vagus nerve. Accordingly, an embodiment of the invention that increases stimulus energy with the patient's heart rate will result in a heart rate that increases at a much slower rate, as reflected by the lower curve 174 in FIG. 13. The slope of the curve 174 is smaller than the slope of the curve 170 (for the patient not receiving electrical stimulus). The slope of the curve 174 is also lower than the slope of the curve 172 (for an embodiment that delivers a constant amount of electrical stimulus.

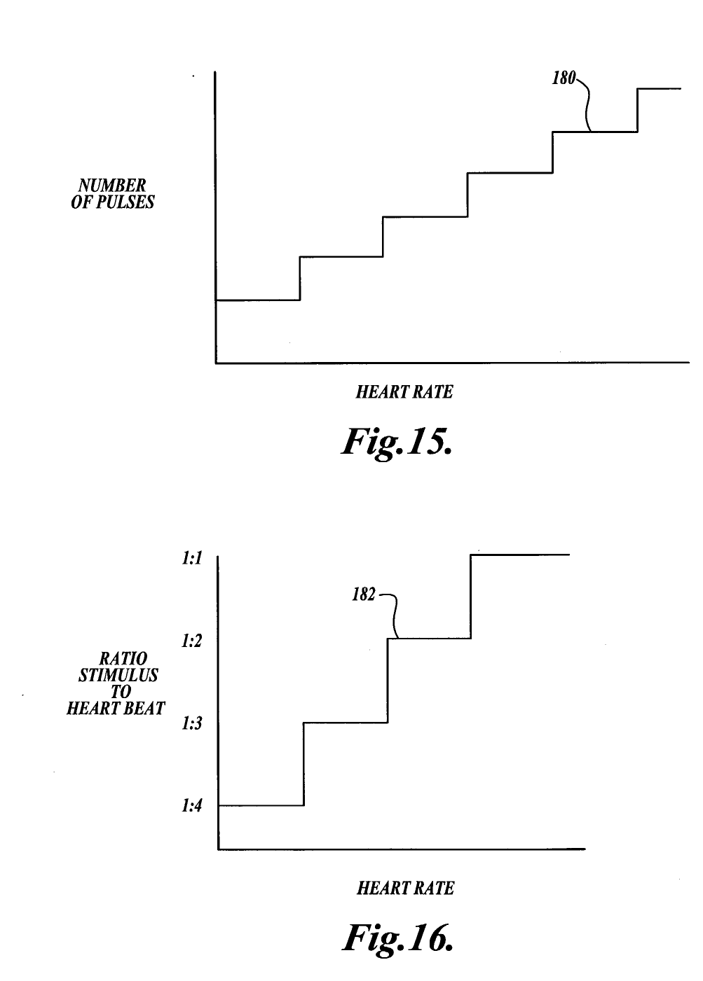

FIGS. 15 and 16 illustrate two exemplary embodiments of the invention in which the amount of energy delivered to the patient varies as a function of the patient's heart rate. In FIG. 15, the curve 180 indicates an increasing number of pulses in the electrical stimulus delivered to the patient as the patient's heart rate increases. The curve 180 is shown in a stepped form to reflect the number of pulses delivered to the patient as a function of the patient's heart rate. The actual number of pulses and the increase in number of pulses for each period of stimulation may vary according to patient circumstances, and thus are not numerically set forth in FIG. 15. For example, for one patient, each stimulus may be comprised of five pulses for a heart rate between 85 bpm and 90 bpm, seven pulses for a heart rate of 90-95 bpm, 10 pulses for a heart rate of 95-100 bpm, etc. For another patient, each stimulus may be comprised of 10 pulses for a heart rate of 80-87 bpm, 12 pulses for a heart rate of 87-95 pbm, 16 pulses for a heart rate of 95-100 bpm, etc. The present invention is not limited by the number of pulses nor the increase in number of pulses for any particular heart rate. After evaluating a patient's condition, a physician may program the device 10 to operate in accordance with a curve 180 using the telemetry features discussed above.

An alternative embodiment of the invention may increase the amount of stimulus energy delivered to the patient by decreasing the stimulus to heart beats ratio. As noted earlier, the present invention does not require delivery of an electrical stimulus during a refractory period of every heart beat. As shown by the curve 182 in FIG. 16, for a lower heart rate, the device 10 may deliver one period of electrical stimulus for every four heart beats. As the patient's heart rate increases, the ratio may be reduced to one period of stimulus for every three heart beats. The ratio may eventually be reduced such that a period of stimulus is delivered after every heart beat (i.e., a 1:1 ratio). The curve 182 in FIG. 16 is exemplary only, and does not limit the present invention. Other embodiments of the invention may start with one period of stimulus for every 10 heart beats, for example, with a reduction in the ratio as the patient's heart rate increases.

Both of the curves 180 and 182 in FIGS. 15 and 16 demonstrate the operation of a device in which stimulus is delivered to the patient without a target heart rate. Those with ordinary skill in the art will recognize that a numerical calculation of the heart rate is not necessary in this regard. For example, the detection of a P-wave (or R-wave) may trigger a timer circuit (analog or digital) that causes a stimulus to be delivered during the next refractory period if the interval between P-waves (or R-waves) is too long. At a very basic level, in fact, a device constructed according to the invention may not count the detection of P-waves at all and instead deliver a constant amount of electrical energy at a programmed stimulus to heart beat ratio.

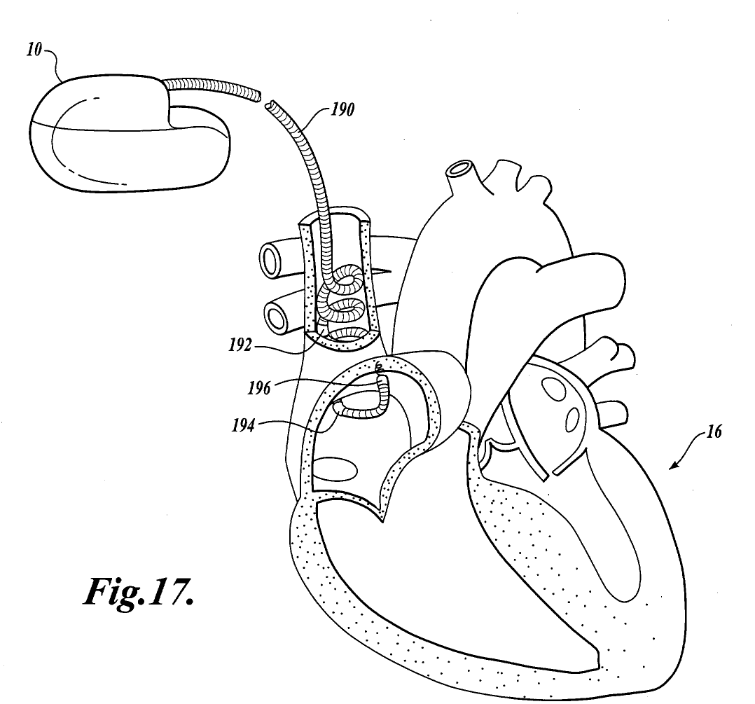

Embodiments of the invention may also include alternative lead configurations such as the helical lead configuration shown in FIG. 17. In FIG. 17, the device 10 is shown connected to a catheter containing an electrical lead 190 that has been implanted in a patient's heart 16. The electrical lead 190, as illustrated, enters the heart 16 through the superior vena cava and is wound in a helical shape. Although multiple windings are shown, the electrical lead 190 may have as few as one winding as it enters the heart 16.

The electrical lead 190 also illustrates the use of more than one electrode in an electrical lead according to the invention. Specifically, the electrical lead 190 includes three electrodes 192, 194, and 196. Preferably, at least two of the electrodes are positioned closely to the tissue in the area of the post-ganglionic right vagus nerves. The positioning of the electrodes is assisted by the helical winding of the lead. As with the lead 12 discussed earlier, the spacing between the electrodes 192, 194, 196 is preferably greater than that which is common in pacing leads so that the electrodes capture a wider area of tissue around the sinoatrial node. The electrical lead 190, as illustrated, also includes a helical screw at the tip to secure the lead to the heart tissue. The ring electrodes 192, 194 and tip electrode 196 may be comprised of conventional components similar to the electrodes in the electrical leads 12 and 14 shown in FIGS. 1A and 1B.

While the above disclosure provides various exemplary embodiments of the invention, it should not be considered limiting with respect to the scope of the invention. The scope of the invention should be determined from the following claims and equivalents thereto.

The embodiments of the invention in which an exclusive property or privilege is claimed are defined as follows:

1. A device for reducing the normal sinus heart rate of a patient, comprising:
(a) a lead with electrodes configured for implantation in the patient such that the electrodes are in electrical communication with tissue proximate to a right vagus nerve of the patient;
(b) detection circuitry with electrodes configured to detect a complex in the patient's normal sinus rhythm;
(c) stimulation circuitry in electrical communication with the lead for delivering electrical energy to the lead electrodes to stimulate the right vagus nerve of the patient; and
(d) control circuitry in communication with the stimulation circuitry for controlling the delivery of the electrical energy to the lead electrodes.

2. The device of claim 1, in which the lead electrodes are also the electrodes for the detection circuitry.

3. The device of claim 1, in which the lead electrodes are configured to be placed in or near an atrium of the patient's heart.

4. The device of claim 3, in which the atrium is the right atrium, and in which the lead electrodes deliver electrical energy that stimulates the patient's right vagus nerve.

5. The device of claim 4, in which the lead electrodes are configured to be placed proximate to the junction of the superior vena cava and the right atrium near the sinoatrial node of the patient.

6. The device of claim 3, in which the detection circuitry is configured to detect a P-wave in the patient's normal sinus rhythm.

7. The device of claim 6, in which the delivery of electrical energy is synchronized with the detection of a P-wave.

8. The device of claim 7, in which the delivery of electrical energy is synchronized to occur during a refractory period following the detection of a P-wave.

9. The device of claim 1, in which the lead is a first lead, the device further comprising a second lead with electrodes implanted in a ventricle of the patient's heart.

10. The device of claim 9, in which the electrodes of the second lead are also the electrodes for the detection circuitry.

11. The device of claim 9, in which the detection circuitry is configured to detect an R-wave in the patient's normal sinus rhythm.

12. The device of claim 1, further comprising an activity sensor in communication with the control circuitry, the activity sensor being configured to sense physical activity of the patient.

13. The device of claim 12, in which the amount of electrical energy delivered to the lead electrodes is adjusted when the activity sensor senses increased physical activity in the patient.

14. The device of claim 1, in which an actual heart rate of the patient is determined from the detection of complexes in the patient's normal sinus rhythm.

15. The device of claim 14, in which the electrical energy is delivered in one or more pulses, and in which the control circuitry is configured to control the voltage of the one or more pulses in accordance with the patient's actual heart rate.

16. The device of claim 14, in which the electrical energy is delivered in one or more pulses, and in which the control circuitry is configured to control the duration of the one or more pulses in accordance with the patient's actual heart rate.

17. The device of claim 14, in which the electrical energy is delivered in one or more pulses, and in which the control circuitry is configured to control the number of pulses in accordance with the patient's actual heart rate.

18. The device of claim 14, in which the control circuitry is configured to control the ratio of stimulus to heart beats in accordance with the patient's actual heart rate.

19. The device of claim 1, in which the control circuitry is configured to control the delivery of electrical energy to the lead electrodes in accordance with a difference between an actual heart rate of the patient and a target heart rate for the patient.

20. The device of claim 19, in which the control circuitry is further configured to measure day information and to automatically adjust the target heart rate for the patient based on current day information.

21. The device of claim 20, in which the current day information reflects the number of days since the control circuitry began to measure day information.

22. The device of claim 21, in which the current day information is measured from when the lead was implanted in the patient.

23. The device of claim 20, in which the current day information reflects an amount of time since the control circuitry began to measure day information.

24. The device of claim 19, in which the control circuitry is further configured to measure time of day information and to automatically adjust the target heart rate for the patient based on current time of day information.

25. The device of claim 24, in which the control circuitry is configured to automatically adjust the target heart rate downward when the current time of day information reflects a time in which the patient is normally sleeping.

26. The device of claim 19, further comprising an activity sensor in communication with the control circuitry, the activity sensor being configured to sense physical activity of the patient.

27. The device of claim 26, in which the control circuitry is configured to automatically adjust the target heart rate for the patient based on the physical activity sensed in the patient.

28. The device of claim 19, in which the electrical energy is delivered in one or more pulses, and in which the control circuitry is configured to control the voltage of the one or more pulses in accordance with the difference between the patient's actual heart rate and the target heart rate.

29. The device of claim 19, in which the electrical energy is delivered in one or more pulses, and in which the control circuitry is configured to control the duration of the one or more pulses in accordance with the difference between the patient's actual heart rate and the target heart rate.

30. The device of claim 19, in which the electrical energy is delivered in one or more pulses, and in which the control circuitry is configured to control the number of pulses in accordance with the difference between the patient's actual heart rate and the target heart rate.

31. The device of claim 19, in which the control circuitry is configured to control the ratio of stimulus to heart beats in accordance with the difference between the patient's actual heart rate and the target heart rate.

32. The device of claim 1, in which the electrical energy is delivered to the lead electrodes over a period of days to gradually reduce the normal sinus heart rate of the patient.

33. The device of claim 1, in which the electrical energy delivered to the lead electrodes is increased over a period of days to gradually reduce the normal sinus heart rate of the patient.

34. A device for treating heart failure or ischemia in a patient, comprising:
(a) a power source for providing electrical energy;
(b) a stimulator connected to the power source for delivering electrical energy in one or more pulses shaped for stimulation of the right vagus nerve of the patient; and
(c) a processor in communication with the stimulator, the processor being configured to control the delivery of the one or more pulses from the stimulator, wherein the stimulation of the right vagus nerve by the one or more pulses produces a reduction of the normal sinus heart rate of the patient.

35. The device of claim 34, in which the processor is comprised of basic logic circuitry that causes the stimulator to deliver the one or more pulses of electrical energy.

36. The device of claim 34, further comprising detection circuitry configured to receive an electrogram signal from the patient and produce a control signal identifying the detection of a complex in the electrogram signal.

37. The device of claim 36, in which the detection circuitry is a P-wave detector that produces a control signal identifying the detection of a P-wave in the electrogram signal.

38. The device of claim 37, in which the control signal is delivered to the processor which causes the stimulator to deliver the one or more pulses synchronized with the detection of a P-wave.

39. The device of claim 38, in which the delivery of the one or more pulses is synchronized to occur during a refractory period following the detection of a P-wave.

40. The device of claim 37, further comprising a counter for counting the number of P-waves with which stimulation of the right vagus nerve occurred.

41. The device of claim 37, further comprising a counter for counting the number of P-waves with which no stimulation of the right vagus nerve occurred.

42. The device of claim 36, in which the detection circuitry is an R-wave detector that produces a control signal identifying the detection of an R-wave in the electrogram signal.

43. The device of claim 36, in which an actual heart rate of the patient is determined from the detection of complexes in the electrogram signal.

44. The device of claim 43, in which the processor is configured to control the voltage of the one or more pulses in accordance with the patient's actual heart rate.

45. The device of claim 43, in which the processor is configured to control the duration of the one or more pulses in accordance with the patient's actual heart rate.

46. The device of claim 43, in which the processor is configured to control the number of pulses in accordance with the patient's actual heart rate.

47. The device of claim 43, in which the processor is configured to control the ratio of stimulus to heart beats in accordance with the patient's actual heart rate.

48. The device of claim 43, in which the processor is configured to control the delivery of the one or more pulses in accordance with a difference between the actual heart rate and a target heart rate for the patient.

49. The device of claim 48, in which the processor is further configured to automatically adjust the target heart rate for the patient based on a programmed heart rate reduction curve.

50. The device of claim 48, in which the processor is further configured to automatically adjust the target heart rate for the patient based on time of day.

51. The device of claim 48, further comprising an activity sensor in communication with the processor, the activity sensor being configured to sense physical activity of the patient.

52. The device of claim 51, in which the processor is configured to automatically adjust the target heart rate for the patient based on physical activity sensed in the patient.

53. The device of claim 48, in which the processor is configured to control the voltage of the one or more pulses in accordance with the difference between the patient's actual heart rate and the target heart rate.

54. The device of claim 48, in which the processor is configured to control the duration of the one or more pulses in accordance with the difference between the patient's actual heart rate and the target heart rate.

55. The device of claim 48, in which the processor is configured to control the number of pulses in the one or more pulses in accordance with the difference between the patient's actual heart rate and the target heart rate.

56. The device of claim 48, in which the processor is configured to control the ratio of stimulus to heart beats in accordance with the difference between the patient's actual heart rate and the target heart rate.

57. The device of claim 34, in which stimulation is delivered to the patient over a period of days to gradually reduce the normal sinus heart rate of the patient.

58. The device of claim 34, in which the stimulation delivered to the lead electrodes is increased over a period of days to gradually reduce the normal sinus heart rate of the patient.

59. A vagal nerve stimulator, comprising:
(a) electrodes configured for implanting in a patient such that the electrodes are proximate to tissue next to or surrounding the patient's right vagus nerve in the area of the sinoatrial node of the patient's heart;
(b) a detector for receiving a physiological signal from the patient and producing a detection signal signifying detection of a contraction in the patient's heart;
(c) a stimulator configured to receive the detection signal and deliver electrical energy to the electrodes during a refractory period following the contraction of the patient's heart.

60. The vagal nerve stimulator of claim 59, in which the detector is configured to detect a P-wave and produce a detection signal signifying detection of a contraction of an atrium in the patient's heart.

61. The vagal nerve stimulator of claim 60, in which delivery of electrical energy is synchronized with the detection of a P-wave.

62. The vagal nerve stimulator of claim 59, in which the detector is configured to detect an R-wave and produce a detection signal signifying detection of a contraction of an ventricle in the patient's heart.

63. The vagal nerve stimulator of claim 59, in which the stimulator is configured to determine the patient's heart rate from the detection signal and control the voltage of the electrical energy delivered to the electrodes in accordance with the patient's heart rate.

64. The vagal nerve stimulator of claim 59, in which the stimulator is configured to determine the patient's heart rate from the detection signal and control the duration of the electrical energy delivered to the electrodes in accordance with the patient's heart rate.

65. The vagal nerve stimulator of claim 59, in which the stimulator is configured to determine the patient's heart rate from the detection signal and control the ratio of stimulus to heart beats in accordance with the patient's heart rate.

66. A method for reducing the normal sinus heart rate of a patient, comprising:
(a) implanting a lead with electrodes in the patient such that the electrodes are in electrical communication with tissue proximate to a right vagus nerve of the patient;
(b) detecting a complex in the patient's normal sinus rhythm;
(c) stimulating the right vagus nerve of the patient by delivering electrical energy to the lead electrodes synchronized with the detection of a complex in the patient's normal sinus rhythm.

67. The method of claim 66, further comprising placing the lead electrodes in or near an atrium of the patient's heart.

68. The method of claim 67, in which the atrium is the right atrium, and in which the electrical energy stimulates the patient's right vagus nerve in the vicinity of the sinoatrial node and superior vena cava of the patient.

69. The method of claim 66, in which the complex is a P-wave in the patient's normal sinus rhythm and the delivery of electrical energy is synchronized with the detection of a P-wave.

70. The method of claim 69, in which the delivery of electrical energy is synchronized to occur during a refractory period following the detection of a P-wave.

71. The method of claim 66, in which the lead is a first lead, the method further comprising implanting a second lead with electrodes in a ventricle of the patient's heart.

72. The method of claim 71, further comprising detecting an R-wave in the patient's normal sinus rhythm.

73. The method of claim 66, further comprising sensing physical activity of the patient and increasing the amount of electrical energy delivered to the lead electrodes when increased physical activity is sensed in the patient.

74. The method of claim 66, further comprising detecting an actual heart rate of the patient from the detection of complexes in the patient's normal sinus rhythm.

75. The method of claim 73, in which the electrical energy is delivered in one or more pulses, the method further comprising controlling the voltage of the one or more pulses in accordance with the patient's actual heart rate.

76. The method of claim 73, in which the electrical energy is delivered in one or more pulses, the method further comprising controlling the duration of the one or more pulses in accordance with the patient's actual heart rate.

77. The method of claim 73, in which the electrical energy is delivered in one or more pulses, the method further comprising controlling the number of pulses in accordance with the patient's actual heart rate.

78. The method of claim 73, further comprising controlling the ratio of stimulus to heart beats in accordance with the patent's actual heart rate.

79. The method of claim 66, further comprising controlling the delivery of electrical energy to the lead electrodes in accordance with a difference between an actual heart rate of the patient and a target heart rate for the patient.

80. The method of claim 79, further comprising measuring day information and automatically adjusting the target heart rate for the patient based on current day information.

81. The method of claim 80, in which the current day information reflects the number of days since beginning to measure day information.

82. The method of claim 81, in which the current day information is measured from when the lead was implanted in the patient.

83. The method of claim 80, in which the current day information reflects an amount of time since beginning to measure day information.

84. The method of claim 79, further comprising measuring time of day information and automatically adjusting the target heart rate for the patient based on current time of day information.

85. The method of claim 84, in which the target heart rate is automatically adjusted downward when the current time of day information reflects a time in which the patient is normally sleeping.

86. The method of claim 79, further comprising sensing physical activity of the patient and automatically adjusting the target heart rate for the patient based on physical activity sensed in the patient.

87. The method of claim 79, in which the electrical energy is delivered in one or more pulses, the method further comprising controlling the voltage of the one or more pulses in accordance with the difference between the patient's actual heart rate and the target heart rate.

88. The method of claim 79, in which the electrical energy is delivered in one or more pulses, the method further comprising controlling the duration of the one or more pulses in accordance with the difference between the patient's actual heart rate and the target heart rate.

89. The method of claim 79, in which the electrical energy is delivered in one or more pulses, the method further comprising controlling the number of pulses in accordance with the difference between the patient's actual heart rate and the target heart rate.

90. The method of claim 79, further comprising controlling the ratio of stimulus to heart beats in accordance with the difference between the patient's actual heart rate and the target heart rate.

91. The method of claim 66, further comprising delivering the electrical energy to the lead electrodes over a period of days to gradually reduce the normal sinus heart rate of the patient.

92. The method of claim 66, further comprising delivering the electrical energy the lead electrodes in an amount that is increased over a period of days to gradually reduce the normal sinus heart rate of the patient.

93. A method for treating heart failure or ischemia, comprising:
(a) providing a power source;
(b) delivering electrical energy from the power source in one or more pulses shaped for stimulation of the right vagus nerve of a patient; and
(c) controlling the delivery of the one or more pulses such that the stimulation of the right vagus nerve by the one or more pulses produces a reduction of the normal sinus heart rate of the patient.

94. The method of claim 93, further comprising receiving an electrogram signal from the patient and producing a control signal identifying the detection of a complex in the electrogram signal.

95. The method of claim 94, in which the complex is a P-wave, the method further comprising delivering the one or more pulses synchronized with the detection of a P-wave.

96. The method of claim 95, in which the delivery of the one or more pulses is synchronized to occur during a refractory period following the detection of a P-wave.

97. The method of claim 94, further comprising counting the number of complexes with which stimulation of the right vagus nerve occurred.

98. The method of claim 94, further comprising counting the number of complexes with which no stimulation of the right vagus nerve occurred.

99. The method of claim 94, in which the complex is an R-wave in the electrogram signal.

100. The method of claim 94, further comprising determining an actual heart rate of the patient from the detection of complexes in the electrogram signal.

101. The method of claim 100, further comprising controlling the voltage of the one or more pulses in accordance with the patient's actual heart rate.

102. The method of claim 100, further comprising controlling the duration of the one or more pulses in accordance with the patient's actual heart rate.

103. The method of claim 100, further comprising controlling the number of pulses in accordance with the patient's actual heart rate.

104. The method of claim 100, further comprising controlling the ratio of stimulus to heart beats in accordance with the patient's actual heart rate.

105. The method of claim 100, in which the delivery of the one or more pulses is controlled in accordance with a difference between the actual heart rate and a target heart rate for the patient.

106. The method of claim 105, further comprising automatically adjusting the target heart rate for the patient based on a preprogrammed heart rate reduction curve.

107. The method of claim 105, further comprising automatically adjusting the target heart rate for the patient based on time of day.

108. The method of claim 105, further comprising sensing physical activity of the patient and automatically adjusting the target heart rate for the patient based on physical activity sensed in the patient.

109. The method of claim 105, further comprising controlling the voltage of the one or more pulses in accordance with the difference between the patient's actual heart rate and the target heart rate.

110. The method of claim 105, further comprising controlling the duration of the one or more pulses in accordance with the difference between the patient's actual heart rate and the target heart rate.

111. The method of claim 105, further comprising controlling the number of pulses in the one or more pulses in accordance with the difference between the patient's actual heart rate and the target heart rate.

112. The method of claim 105, further comprising controlling the ratio of stimulus to heart beats in accordance with the difference between the patient's actual heart rate and the target heart rate.

113. The method of claim 93, further comprising delivering the electrical stimulation to the patient over a period of days to gradually reduce the normal sinus heart rate of the patient.

114. The method of claim 1, further comprising delivering the electrical stimulation to the lead electrodes in an amount that is increased over a period of days to gradually reduce the normal sinus heart rate of the patient.

115. A device for treating cardiovascular disease in a patient, comprising:
(a) a lead with electrodes configured for implantation in the patient near the sinoatrial node of the patient's heart; and
(b) a pulse generator in electrical communication with the lead electrodes for delivering one or more electrical pulses to the electrodes that stimulate cholinergic fibers to release acetylcholine which is taken up by the patient's sinoatrial node.

116. The device of claim 115, in which the lead electrodes are configured for placement at the junction of the superior vena cava and the right atrium of the patient's heart.

117. The device of claim 115, in which the acetylcholine that is taken up by the sinoatrial node causes a time delay to the next heart beat in the patient.

118. The device of claim 117, in which the pulse generator controls the time delay to the next heart beat by controlling the electrical pulses delivered to the patient.

119. The device of claim 117, in which increasing the time delay to the next heart beat causes a reduction of blood pressure in the patient.

120. The device of claim 119, in which the reduction of blood pressure is determined from a difference between the patient's current blood pressure and a target blood pressure.

121. The device of claim 115, further comprising detection circuitry configured to detect a heart beat in the patient and cause the pulse generator to deliver an electrical pulse during a refractory period in the heart beat.

122. A method of treating cardiovascular disease in a patient, comprising:
(a) implanting a lead with electrodes in the patient near the sinoatrial node of the patient's heart; and
(b) delivering to the lead electrodes one or more electrical pulses that stimulate cholinergic fibers to release acetylcholine which is taken up by the patient's sinoatrial node.

123. The method of claim 122, in which the lead electrodes are implanted at the junction of the superior vena cava and the right atrium of the patient's heart.

124. The method of claim 122, in which the acetylcholine that is taken up by the sinoatrial node causes a time delay to the next heart beat in the patient, the method further comprising controlling the time delay to the next heart beat by controlling the electrical pulses delivered to the patient.

125. The method of claim 124, further comprising causing in a reduction of blood pressure in the patient by increasing the time delay to the next heart beat.

126. The method of claim 125, further comprising determining the reduction of blood pressure from a difference between the patient's current blood pressure and a target blood pressure.

127. The device of claim 122, further comprising detecting a heart beat in the patient and delivering an electrical pulse to the lead electrodes during a refractory period in the heart beat.

128. An electrical lead configured for implantation in a patient's heart, comprising:
(a) a length of electrically conductive material in an insulating substrate; and
(b) a plurality of electrodes attached to the insulating substrate and electrically connected to the conductive material, in which a first electrode is disposed along the length of the insulating substrate and a second electrode is disposed near a distal end of the lead, the first and second electrodes having sufficient distance between them to allow the first electrode to be located in the superior vena cava while the second electrode is located in the atrium near the sinoatrial node when implanted in the heart of the patient.

129. The electrical lead of claim 128, further comprising a hook-shaped bend in the insulating substrate between the first and second electrodes.

130. The electrical lead of claim 128, further comprising one or more helical windings of the insulating substrate that places the first electrode in the superior vena cava and the second electrode near the sinoatrial node when the lead is implanted in the heart of the patient.

131. The electrical lead of claim 128, in which the lead includes three or more electrodes attached to the insulating substrate and electrically connected to the conductive material, wherein when implanted, all of the electrodes are disposed proximal to tissue containing right vagus nerves that innervate the sinoatrial node of the patient.
